(19) United States
(12) Patent Application Publication
(10) Pub. No.: US 2009/0005745 A1
(43) Pub. Date: Jan. 1, 2009

(54) Transdermal drug delivery systems for delivering anti-inflammatory drugs

(76) Inventors: Jie Zhang, Salt Lake City, UT (US); Kevin S. Warner, West Jordan, UT (US)

Correspondence Address: THORPE NORTH & WESTERN, LLP., P.O. Box 1219, SANDY, UT, 84091-1219, US

(21) Appl. No.: 12/156,473

(22) Filed: May 30, 2008

Related U.S. Application Data
(60) Provisional application No. 60/932,841, filed on Jun. 1, 2007.

(51) Int. Cl. A61K 9/70; A61F 7/08

(52) U.S. Cl. 604/291; 424/449; 607/96

(57) ABSTRACT

Systems for delivering an anti-inflammatory drug and methods for treating osteoarthritis are provided. Such systems can comprise, in one embodiment, a transdermal patch with a sufficient amount of anti-inflammatory drug in a formulation for sustained transdermal delivery at a human skin site. The system can further include a permeation composition or device, such as a heating device. The heating device can be configured for application over the transdermal patch and the human skin site. Further, the heating device can be configured for heating the human skin site to a specific temperature range from 36° C. to 42° C., and for maintaining the human skin site within that temperature range for a period of at least 4 or 5 hours.

This application claims the benefit of U.S. Provisional Patent Application No. 60/932,841, filed Jun. 1, 2007, which is herein incorporated by reference.

### BACKGROUND

Although oral NSAIDs (nonsteroidal anti-inflammatory drugs) have been used effectively to treat musculoskeletal pain and inflammation for decades, they have significant potential to cause bleeding in the gastrointestinal (GI) tract. Such bleeding has been linked with many deaths each year. The potential to cause GI bleeding is believed to increase with increasing concentrations of NSAIDs in the patient's systemic circulation (plasma drug concentrations).

Attempts have been made to deliver anti-inflammatory and analgesic drugs directly into joints and muscles transdermally to treat musculoskeletal pain and inflammation of various causes, such as arthritis induced pain. For example, creams containing NSAIDs (nonsteroidal anti-inflammatory drugs) are marketed in Europe and Japan for treating joint pain.

In accordance with this, it would be desirable to provide systems and methods for administering anti-inflammatory drugs in a manner that is less harmful to the patient, and specifically reduces the destructive effects such drugs have on a patient's GI tract. Further, it would be desirable to administer such drugs in a manner that provides improved dermal drug delivery, as well as provide other additional benefits.

### SUMMARY

The present disclosure is related to a system and a method for delivering certain drugs that combines the effects of transdermal delivery with a heating system, for delivering drugs into regional tissues such as joints and muscles. Such combination can harness the benefits of both more regional drug delivery and the use of heat for treating musculoskeletal pain or inflammation. In order to develop combined drug delivery-heating systems that are efficacious, safe, and easy to use, many properties of the combined system have to be carefully designed, and the present disclosure is also related to those designs.

Accordingly, a system for delivering an anti-inflammatory drug can comprise two major components: a heating device and an anti-inflammatory drug-containing transdermal patch. The transdermal patch can include a sufficient amount of the anti-inflammatory drug to provide sustained delivery of the drug at a human skin site. The heating device can be configured for application over the transdermal patch and the human skin site. Additionally, the heating device can be configured to heat the human skin site to a specific temperature range from 36° C. to 42° C., and can maintain the human skin site within the specific temperature range for a period of at least 5 hours.

In another embodiment, a system for the delivery of ketoprofen can include a transdermal patch having a skin-drug contact area of from 50 to 400 cm². The system can be configured to produce a mean blood plasma concentration in a group of at least 10 human subjects of at least 33 ng/ml within 4 hours after initial application of the transdermal patch to a skin surface of the human subject.

Another system of the present disclosure provides for the delivery of ketoprofen is a system which includes a transdermal patch which has a skin-drug contact area of 50 to 400 cm². The system can be capable of producing a mean blood plasma concentration of ketoprofen produced by the unit skin drug contact area in a group of human subjects of at least ten of at least 0.19 ng/ml/cm² within 4 hours of application to the skin surface the human subjects. Unit skin drug contact area can be defined as the mean plasma concentration of ketoprofen divided by the total drug formulation-skin contact surface area. The phrase “drug formulation-skin contact surface area” is used interchangeably with phrases such as “drug-skin contact area.”

In another embodiment, a system for delivering ketoprofen can include a transdermal patch having a skin-drug contact area and a heating device. The system can be capable of producing a mean blood plasma concentration of ketoprofen in human subjects that does not peak within 7 hours of commencement of the application to the skin surface of human subjects.

The present disclosure provides an additional embodiment in which a system for delivering ketoprofen can include a transdermal patch having a skin-drug contact area and a heating device. The system can be capable of producing a mean blood plasma concentration of ketoprofen in human subjects that exceeds about 100 ng/mL within 7 hours of commencement of application to the skin surface of human subjects.

Another embodiment of the present disclosure provides for a system for delivering ketoprofen. The system includes a transdermal patch having a skin-drug contact area and a heating device. The system can be capable of producing a mean blood plasma concentration of ketoprofen in human subjects that exceeds about 120 ng/mL within 8 hours of commencement of application to the skin surface of human subjects.

Diclofenac can alternatively be administered according to embodiments of the present disclosure. In one aspect, a system for delivery can include a transdermal patch capable of producing a blood plasma concentration of diclofenac per unit of the skin-drug contact area within 4 hours of application to a skin surface of a human subject of at least 0.08 ng or 0.11 ng diclofenac/mL/cm². In another embodiment, a system for delivery can include a transdermal patch capable of producing a blood plasma concentration of diclofenac per unit of the skin-drug contact area within 8 hours of application to a skin surface of a human subject of at least 0.23 ng diclofenac/mL/cm².

Similarly, another embodiment provides for a system for delivering diclofenac which can include a transdermal patch having a skin-drug contact area and a heating device. The system can be capable of producing a mean blood plasma concentration of diclofenac produced by the unit skin drug contact area in human subjects within 4 hours of application to the skin surface of human subjects of at least about 16 ng/mL and 0.1 ng/mL/cm², respectively.

The present disclosure also provides for a method for treating osteoarthritis. The method includes placing a transdermal patch at a human skin site adjacent to a joint suffering from osteoarthritis. The transdermal patch can include a sufficient amount of an anti-inflammatory drug for sustained transdermal delivery. The transdermal patch and human skin can be heated with a heating device. The heating device can be configured to heat the human skin site to a specific temperature range from 36° C. to 42° C., and then maintain the human skin site within the specific temperature range for a period of at least 5 hours.

Additional features and advantages of the disclosure will be apparent from the following detailed description, which illustrates, by way of example, features of the disclosure. For example, optionally, many of the above systems can benefit from the use of a permeation enhancing means such as component suitable for generating heat, iontophoresis, radiation, ultrasound, phase transition of supersaturated solutions, chemical enhancement means, etc.

### BRIEF DESCRIPTION OF THE DRAWINGS

FIG. 1 is a schematic representation of a system for dermal delivery of a drug, in accordance with one embodiment of the present disclosure.

FIGS. 2A and 2B are a schematic representation of an alternative system for dermal delivery of a drug, in accordance with another embodiment of the present disclosure.

FIG. 3 is a single exemplary top view of a system shown schematically in FIGS. 1, 2A, and 2B.

FIG. 4 is a graph of the plasma concentration results of an experiment wherein identical compositions were administered transdermally, with and without a heating device.

FIG. 5 is a graph that shows the skin temperature profiles of human skin generated using a heating device in accordance with embodiments of the present disclosure.

FIG. 6 is a graph that shows the skin temperature profiles of human skin generated using the heating device used in FIG. 5, but with a different patient group under various testing conditions.

### DETAILED DESCRIPTION

Before the present invention is disclosed and described, it is to be understood that this disclosure is not limited to the particular process steps and materials disclosed herein because such process steps and materials may vary somewhat. It is also to be understood that the terminology used herein is used for the purpose of describing particular embodiments only. The terms are not intended to be limiting because the scope of the present disclosure is intended to be limited only by the appended claims and equivalents thereof.

As used in this specification and the appended claims, the singular forms “a,” “an,” and “the” include plural referents unless the content clearly dictates otherwise.

The terms “controlled heating” and “controlled heat” are defined as heat application that is capable of heating a skin surface to pre-determined narrow temperature range for a predetermined duration. A controlled heating device that can be used in accordance with systems and methods of the present disclosure can be configured to generate heat promptly when activated. Controlled heating can be achieved through special design of the heating device. For example, controlled heating can be achieved through the use of a properly configured heating element(s) including an exothermic chemical composition. Considerations in generating controlled heat with an exothermic heating device assembly include proper ratios and chemical components used, as well as physical constraints put on the chemical components, e.g., limiting air flow or oxygen contact, spatial configuration of individual heating elements, conductivity of materials used with chemical components, etc. In one embodiment, the heating device can provide heat at a temperature greater than body temperature, but less than a temperature that would cause irreversible skin damage, e.g., burn the skin. An exemplary temperature range that can be implemented for use is from about 35° C. to about 47° C. In one embodiment, a more preferred temperature range can be from about 36° C. to 42° C. Other desired temperature ranges include from about 38° C. to 42° C., or from 36° C. to 40° C.

As used herein, the term “active” when referring to a body surface, such as skin, indicates that the body surface regularly undergoes flexing, bending, and/or stretching. Such is the case with nearly all joints. For example, knees, elbows, fingers, neck, etc. Additionally, back muscles are considered active body surfaces because of the large amount of flexing, bending, and/or stretching. Areas of the skin that are not regularly stretched during normal activity are not considered to be “active.” For example, the scalp, arms and legs (other than at or near joints), etc., are not considered active body surfaces.

As used herein, the term “foil” refers to a primarily metallic material formed into a thin self-supporting sheet. The foil can comprise any metallic material; however, in one specific embodiment, the material can consist essentially of a metallic material, such as aluminum. Metal alloys are also included within this definition. The term “thin” when referring to a metal foil may be interpreted to mean any metal foil with a thickness from about 0.0001″ (0.1 mil, or 25.4 micrometer) to about 0.01″ (10 mil, or 254 micrometer).

It should be noted that “mean plasma concentration” of any drug described herein is defined as the mean of at least 10 human subjects when the drug is administered under normal test conditions, which is defined as under about 25° C. temperature, over normal intact skin, and on adult human subjects.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, thicknesses, sizes, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 wt % to about 5 wt %” should be interpreted to include not only the explicitly recited values of about 1 wt % to about 5 wt %, but also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

The present disclosure is drawn to systems for delivering anti-inflammatory drugs. In certain specific embodiments, the present disclosure is drawn to systems for delivering ketoprofen and/or diclofenac, particularly for the delivery of ketoprofen into the human knee. The systems include a transdermal patch with an amount of drug for sustained transdermal delivery, and a heating device. In one aspect, the heating device can be configured for application over the transdermal patch and the human skin site. The heating device can further be configured to heat the human skin site to a particular range, and maintain the temperature range for a set amount of time. In another aspect, the heating device can be configured to heat the human skin site and transdermal patch sufficient to produce a blood plasma concentration of the drug that is greater than the transdermal patch alone. In still another aspect, the heating device can be configured to produce a greater blood plasma concentration of the drug area under the curve measurement than without the use of the heating device. Such systems can be used, e.g., for anti-inflammatory drugs such as NSAIDs, diclofenac, COX-2 inhibitors, COX-3 inhibitors, and ketoprofen. Similarly, a method for treating osteoarthritis can include using such a system. Specifically, the method for treating osteoarthritis can include placing a transdermal patch including a sufficient amount of an anti-inflammatory drug for sustained transdermal delivery at a human skin site. The method can further include heating the transdermal patch and human skin with a heating device. The heating device can be configured for heating the human skin to a specific temperature range, and further configured for maintaining that temperature range.

In accordance with the difficulties outlined, various details are provided herein which are applicable to each of the systems for delivering an anti-inflammatory drug generally, the systems for delivering ketoprofen, the systems for delivering diclofenac, and the methods for treating osteoarthritis. Thus, discussion of one specific embodiment is related to and provides support for this discussion in the context of the other related embodiments.

It is also noted that though heat is often described herein as being used to achieve certain blood mean plasma concentrations, in some embodiments, the use of heat is not necessary, as long as the appropriate enhanced blood concentrations can be achieved. For example, chemical enhancement, iontophoresis, infrared radiation, or ultrasound can be used to achieve enhanced blood plasma concentrations in accordance with embodiments of the present disclosure. Thus, heat is described throughout as an exemplary embodiment.

One benefit of the system of the present disclosure is enhanced transdermal drug delivery by controlled heating, as skin permeability to drugs can increase with increasing skin temperature. In addition, the heating itself is also expected to reduce the musculoskeletal pain or inflammation. The combination of the transdermal delivery of a drug and the heat can boost the efficacy of either the drug or the heat alone. Further, in some embodiments, the selection and use of the drug, concentration of drug in a transdermal patch, and/or the amount and duration of the heat can provide synergistic effects.

A system for delivering an anti-inflammatory drug in accordance with embodiments of the present disclosure can include a transdermal patch and a heating device. The transdermal patch can include a drug component. Specifically, the transdermal patch can include a sufficient amount of anti-inflammatory drug in a formulation for sustained transdermal delivery at a human skin site. Such systems can be used on any human skin site, in particular, active body surfaces including knees, elbows, fingers, neck regions, back regions, etc.

A drug component can comprise a formulation that is designed to transdermally deliver the drug. The drug component may also comprise means of affixing itself (or the entire heating-drug combined system in the case of integrated systems) to the skin, such as a layer of adhesive. The formulation can be in the form of a patch, gel, paste, film, powder, oil, emulsion, adhesive, etc. While all of these dosage forms may be used as described herein, a few preferred dosage forms include the drug-in-adhesive patch or a reservoir patch. The drug component may contain one, or a combination, of a variety of therapeutically effective drugs, and optionally, appropriate chemical enhancers. In one embodiment, the drug of choice is an anti-inflammatory drug such as an NSAID, e.g. ketoprofen, diclofenac, salicylates, arylalkanoic acids, profens, fenamic acids, pyrazolidine derivatives, oxicams, COX-2 inhibitors, COX-3 inhibitors, sulphonanilides, licofelone, omega-3 fatty acids, and combinations thereof. In one specific embodiment, the drug can comprise or consist essentially of ketoprofen. In another specific embodiment, the drug can comprise or consist essentially of diclofenac. Still in another specific embodiment, the drug can comprise or consist essentially of a COX-2 inhibitor. In yet another specific embodiment, the drug can comprise or consist essentially of a COX-3 inhibitor. Additionally, other drugs, such as local anesthetics, e.g. lidocaine, could also be beneficially delivered by the systems of the present disclosure. The drug included in the transdermal patch can also include a plurality of anti-inflammatory drugs. For example, the transdermal patch can include ketoprofen and a second anti-inflammatory drug.

In one aspect, the heating device can be configured for application over the transdermal patch and the human skin site. The heating device can be configured for heating the human skin site to a specific temperature range from 36° C. to 42° C., and can further be configured to maintain the human skin site within the specific temperature range for a period of at least 5 hours. In another embodiment, the heating device can be configured for heating a human skin site to a specific temperature range of from about 38° C. to about 42° C. In yet another embodiment, the heating device can be configured for heating a human skin site to a specific temperature range of from about 36° C. to about 40° C.

Maintaining the heated temperature of the human skin site for an extended period of time allows for improved drug delivery during that time. As mentioned, in one aspect, the heating device can be configured to maintain the human skin site within the specific temperature range for at least 5 hours. In some situations, it may be beneficial to have a longer time period of heating. In such cases, the heating device can be configured for to maintain the human skin site within the specific temperature range for at least 6 hours, or even for at least 7 hours. Alternatively, in some situations, depending on the drug used and anticipated application, it may be beneficial to have a specific time wherein the heating device maintains the human skin site in the specific temperature range of 38° C. to 42° C. In such cases, the heating device can be configured to automatically cool to below 38° C. after heating the human skin site for a specific time period. For example, the heating device can be configured to cool to below 38° C. after heating the human skin site for between 5 and 10 hours, e.g., after 5 hours, after 6 hours, or after 7 hours, within the specific temperature range.

A heating device for use in accordance with embodiments of the present disclosure can generate and provide heat by one of a number of mechanisms. One mechanism involves generating heat chemical-based reaction. Such chemical-based reaction can include an exothermic chemical reaction. A non-limiting example of an exothermic chemical reaction that can be used is an oxidation reaction. Examples of oxidation reactions include an oxidation reaction of an alcohol having one to four carbons, and the oxidation of certain metals. Heat can be generated, for example, by oxidation of certain metals, such as iron, through the use of an exothermic chemical composition. Such a mechanism can be configured to generate heat by an oxidation reaction between a component, e.g., iron, within the heating device and oxygen in ambient air. U.S. Pat. No. 6,756,053, which is incorporated herein by reference in its entirety, describes such heating devices. Other heating mechanisms can also be used, such as heating by phase transition (such as phase transition of sodium acetate solutions) and electricity.

The amount of exothermic chemical composition in a heating device can vary from design to design. It can be desirable to limit the amount of chemical composition in the heating device, as a large amount of chemical composition can be cumbersome and can inherently limit the potential uses of a drug delivery apparatus. Such is the case with skin that regularly experiences flexing, stretching, bending, or other movement. In one aspect, the heating device can include no more than 35 grams of an exothermic chemical composition and can be configured to heat an area of skin greater than about 40 cm², or in another embodiment, greater than about 80 cm². In another embodiment, an exothermic chemical composition can produce all heat for the heating device.

Although heat in the heating device can be generated by various mechanisms, in one aspect, formulations can utilize an exothermic oxidation reaction of metal. The heating device, therefore, can include metal powder. Non-limiting examples of metal particulates, e.g., powders or filings, which can be used in the heating device includes iron and aluminum. As discussed, the heating device can also have multiple heating elements, each containing an exothermic composition. An exothermic chemical composition can further include activated carbon, salt (such as sodium chloride), and water. In one aspect, a water-retaining substance, such as vermiculite, can be included in the composition. Depending on the configuration of the heating device, one issue with the exothermic chemical composition can be that during the long storage time, gas (believed to be methane and hydrogen) is generated which puffs up an air tight container of the heating device (or the container containing an integrated heating and drug components), which can, in some cases, pose problems in storage and transportation. Certain amounts of sulfur-containing compounds, or salt thereof, such as elemental sulfur, sulfates, sulfites, sulfides, or thiosulfates, can reduce or eliminate this gas generation problem when included in the packaging.

Water content in the exothermic chemical composition can have an impact on the heating temperature profile of the heating device. The weight ratio of water to the rest of the ingredients can be in the range of about 1:2.6 to about 1:5.0. It has been discovered in accordance with embodiments of the present disclosure that if the weight ratio of water to the rest of the ingredients is outside these ranges, the heating profiles (temperature, duration) are less desirable, though ranges outside of these are included within the scope of the present disclosure to the extent that they are functional.

In one specific embodiment, the exothermic chemical reaction of the heating device can be controlled by holes in an air-impermeable cover, thus regulating oxygen flow. In one aspect, the heating device can include a plurality of individual heating elements. Each heating element can comprise a pre-formed bag formed of a material(s) that is substantially freely permeable to air and water. The heat generating composition resides inside the bag. In some aspects, each heating element can be formulated as part of a chambered heating element having a cover and having a certain number of holes associated therewith, e.g. located directly above.

In one aspect, the heat-generating composition in the heating device can have access to ambient oxygen only through the holes in a cover that is made of air-impermeable material. In this way, the flow rate of oxygen from ambient air into the heat generating composition, which is one of the factors that can determine the heating temperature in such heat device configuration, is controlled by the size and number of holes on the cover.

In accordance with the present disclosure, the transdermal patch can have a skin contact area where the anti-inflammatory drug is delivered to the skin. In one aspect, the skin contact area can be at least 40 cm² and the heating device can have a weight of less than 100 g. Further, the heating device can have a weight of less than 60 g, or even 40 g. In an alternate embodiment, the skin contact area can be at least 80 cm² and the heating device can have a weight that is less than about 100 g.

Another system for dermal drug delivery can include a heating device with at least one heating element, and a similar drug containing layer with a surface area of about 50 cm² to about 400 cm². In one embodiment, the surface area of the drug containing layer can be about 100 cm² to about 250 cm². The heating device can be physically integrated and thermally associated with the drug containing layer. Further, the heating device and drug containing layer can be configured to remain adhered to a skin surface for an extended time when subjected to stretching and movement. Such stretching is the type often encountered with joints such as the knee. The transdermal patch can be in contact with the human skin site for a substantially continuous period of about 4 hours to about 18 hours, or about 5 hours to about 14 hours.

The drug delivery apparatus and the heating device in particular, can be of any shape and size. The heating device can be designed to heat an area of skin greater than about 60 cm², 80 cm², 100 cm², and even greater than 120 cm². Preferably, the temperature variation along the skin surface area in thermal contact with the heating device is minimized. Drug delivery can be more consistent from a drug delivery device with minor to no temperature variation across the area of heated skin. In a specific embodiment, the heating device can be configured to heat an area of skin greater than about 40 cm², and produce a heat variation within the area of skin surface covered by the heating device of less about 4° C. while maintaining the human skin site within the specific temperature range for a period of at least 5 hours. In another specific embodiment, the heating device can be configured to heat an area of skin greater than about 80 cm² and produce a heat variation within the area of skin surface covered by the heating device of less about 4° C. while maintaining the human skin site within the specific temperature range for a period of at least 5 hours. In still another specific embodiment, the heating device can be configured to heat an area of skin greater than about 40 cm² and produce a heat variation within the area of skin surface covered by the heating device of less about 4° C. while maintaining the human skin site within the specific temperature range for a period of at least 7 hours. In yet another embodiment, the heating device can be configured to heat an area of skin greater than about 80 cm² and produce a heat variation within the area of skin surface covered by the heating device of less about 4° C. while maintaining the human skin site within the specific temperature range for a period of at least 5 hours.

For the systems of this disclosure that include a drug component and a heating device, it is desirable to have a sufficient drug-skin contact area and heating surface area while the system is not too heavy. Too heavy a system on the patient can cause discomfort and higher likelihood of separation from the skin. In some embodiments of the present disclosure, the drug-skin contact area is at least about 100 cm², or at least about 150 cm², while the total weight of the entire system is less than 45 grams. The heated areas in those embodiments are no less than 40 cm², and preferably no less than 60 cm².

In one embodiment, the heating device can comprise a plurality of discrete heating elements. In a specific embodiment, the heating device can comprise from 2 to 10, or usually 3 to 7 heating elements. In a specific embodiment, each heating member can comprise a pre-formed bag or pouch formed of a material(s) that is substantially freely permeable to air and water. A chemical mixture capable of reacting exothermically can be contained in the pouches. In some aspects, each heating element can be formulated as part of a chambered heating device having a cover and having a certain number of holes associated therewith. Heating elements can be arranged in any manner that is conducive to providing heat to the system. In one aspect, the arrangement can be unstructured. In another embodiment, the heating elements can be formed into one or more rows. In more specific embodiments, the heating elements can be arranged into one, two, or three or more rows. In still another embodiment, the heating elements are arranged in pattern that is non-linear. For example, it may be desirable to arrange the heating elements in a manner that is ergonomically configured for application over a specific joint. For example, without limitation, a knee or elbow joint may benefit from radially positioned heating elements that surround the knee cap or elbow.

In accordance with the present disclosure, the heating device can be configured for heating the human skin site and the transdermal patch for a period of time sufficient to generate blood plasma concentrations of the anti-inflammatory drug 2, 4, or 6 hours after the application of the transdermal patch that is at least 40% greater than the blood plasma concentration would be after application of the transdermal patch for the same period of time without the use of the heating device or other permeation enhancing scheme.

In the present disclosure, although the target tissues are joints and muscles rather than the systemic circulation, drug concentrations in the systemic circulation can be used to gauge the rate or amount of drug permeated across the skin. Use of systemic circulation as a gauge is useful because 1) it is much more difficult to measure drug concentrations in local muscle tissues and joints and 2) for a given drug-skin contract area, plasma drug concentrations are expected to be approximately proportional to the skin permeability.

For a transdermal drug targeted for local or regional tissues, such as joints and/or muscles, it is particularly desirable to be able to deliver sufficient amount of the drug, such as ketoprofen, diclofenac, COX inhibitors, etc., across the finite skin area adjacent to the target tissues. This is because the drug molecules that permeate across the skin area adjacent to the targeted local tissues have the highest portions going into the target tissues, and thus, can produce the highest desired clinical results to adverse side effect ratio. NSAID drug molecules that permeate across the skin far away from the targeted tissues have little chance to enter the targeted tissues directly and will end up primarily in the systemic circulation, which provides a low therapeutic effect to adverse side effect ratio. This property is unique from conventional drug delivery systems whose target is systemic circulation, because in those systems, drug permeating across skin areas anywhere will end up in the systemic circulation and contribute to the desired effect(s).

Therefore, in conventional systemic transdermal drug delivery systems, total drug absorption by the entire skin-drug contact area is more important than the drug absorption per unit of the skin-drug contact area. The concentration per unit of skin-drug contact area is defined as the plasma drug concentration produced by a transdermal drug delivery system divided by its skin-drug contact area. Therefore, the plasma drug concentration produced per unit of the skin-drug contact area is a valuable parameter of the system to consider. A typical unit of this parameter is ng/mL/cm². For example, if the mean plasma ketoprofen concentration at 8 hours is approximately 125 ng/mL and the drug-skin contact area is 100 cm², the plasma drug concentration produced by a unit of skin-drug contact area is 1.25 ng/ml/cm² at 8 hours. This being said, by placing the transdermal patch over or proximate to the joint that is being treated, much of the drug will pass into and through the target joint, requiring less drug for treatment.

In another embodiment of the present disclosure, a system for delivering ketoprofen can comprise a transdermal patch having a skin-drug contact area and a heating device that is capable of producing a ketoprofen concentration per unit of the skin-drug contact area mean of at least about 0.15 ng/mL/cm², at least about 0.19 ng/mL/cm², or even at least about 0.23 ng/mL/cm² within 4 hours of application to a skin surface of a human subject.

In another embodiment of the present disclosure, a system for delivering ketoprofen can comprise a transdermal patch having a skin-drug contact area and a heating device that is capable of producing a ketoprofen concentration per unit of the skin-drug contact area mean of at least about 0.35 ng/mL/cm², or at least about 0.38 ng/mL/cm², or even at least about 0.46 ng/mL/cm² within 6 hours of application to a skin surface of a human subject.

In another embodiment of the present disclosure, a system for delivering ketoprofen can comprise a transdermal patch having a skin-drug contact area and a heating device that is capable of producing a mean ketoprofen concentration per unit of the skin-drug contact area of at least about 0.47 ng/mL/cm², or at least about 0.56 ng/mL/cm², within about 8 hours of application to a skin surface of a human subject.

It is noted that though the generation of heat though oxidation (such as oxidation of metal powder) is described herein in greater detail, the heating device in the aforementioned systems can be by radiation (microwave or infrared, for example), electricity-resistor means, phase transition of supersaturated solutions, combinations thereof, and/or other heating sources. In further detail, the heating device, for example, can be an electric heating device. Such electric heating device can be powered by a variety of sources, for example battery and/or alternating electric current. Electric devices can be configured to provide a predetermined heating profile so that the heating profile is met automatically after engaging or turning on the electric device, e.g., use of timers, programmed electricity supply, finite batter power, etc. Alternatively, the heating profile can be met merely by providing heat at an appropriate temperature with an instruction to the user to remove the heating device after a specific period of time.

Another property of the present disclosure is the ability to achieve a sustained increase in plasma drug concentrations produced by controlled heating or other means, which is a reflection of more drug delivered across the skin per unit area for longer periods of time. In accordance with this, in one embodiment, a system for delivering ketoprofen can comprise a transdermal patch having a skin-drug contact area and a heating device that is capable of producing a mean plasma concentration of ketoprofen that does not peak until at least about 7 hours, and often at least about 8 hours, after the commencement of the application to the skin of human subjects.

In another embodiment, a system for delivering ketoprofen can comprise a transdermal patch having a skin-drug contact area of 250 cm² or less and a heating device that is capable of producing a mean plasma concentration of ketoprofen that exceeds about 75 ng/mL in about 7 hours after the commencement of the application to the skin of human subjects. In another embodiment of the present disclosure, a system for delivering ketoprofen can comprise a transdermal patch having a skin-drug contact area of 250 cm² or less and a heating device that is capable of producing a mean plasma concentration of ketoprofen that exceeds about 94 ng/mL about 8 hours after the commencement of the application to the skin of human subjects.

Thus, in one aspect of the present disclosure, the ketoprofen blood plasma concentration 2 hours after the application of the system can be at least 50%, 100%, 150%, or even 200% greater than the blood plasma concentration would be after application of the transdermal patch for the same period of time without the use of the heating device. Likewise, the ketoprofen blood plasma concentration 4 hours after the application of the system can be at least 50%, 100%, and even 150% greater than the blood plasma concentration would be after application of the transdermal patch for the same period of time without the use of the heating device. Similarly, the ketoprofen blood plasma concentration 6 hours after application can be at least 50% or 100% greater. In one embodiment, the ketoprofen blood plasma concentrations after any two or all three of 2 hours, 4 hours, and/or 6 hour time points can both or all be at least 40% greater, 80% greater, or 100% greater. In a specific embodiment, the ketoprofen blood plasma concentration after 2 hours can be at least 200% greater, after 4 hours at least 150% greater, and after 6 hours at least 80% greater than the blood plasma concentrations would be after application of the transdermal patch for the same periods of time without the use of the heating device.

In still another aspect of the present disclosure, the heating device can be configured for heating the human skin site and the transdermal patch for a period of time that generates ketoprofen blood plasma concentration area under the curve measurement for time 0 to time 6 hours that is at least 40%, 60%, 80%, or 100% greater than the blood plasma concentration area under the curve measurement would be after application of the transdermal patch for a same period of time without the use of the heating device. Likewise, the ketoprofen blood plasma concentration area under the curve measurement for time 0 to time 4 hours can be at least 50%, 100%, 150%, 200%, or 250% greater than without the use of the heating device.

Non-limiting examples of permeation enhancing components are heat, iontophoresis, radiation (infrared, microwave, etc.), ultrasound, and combinations thereof. Additionally, the ketoprofen delivery system can include a heating device configured for application over the transdermal patch.

Diclofenac can alternatively be administered according to embodiments of the present disclosure. In one aspect, a system for delivery can include a transdermal patch capable of producing a blood plasma concentration of diclofenac per unit of the skin-drug contact area within 4 hours of application to a skin surface of a human subject of at least 0.08 ng or 0.11 ng diclofenac/mL/cm². In another embodiment, a system for delivery can include a transdermal patch capable of producing a blood plasma concentration of diclofenac per unit of the skin-drug contact area within 8 hours of application to a skin surface of a human subject of at least 0.23 ng diclofenac/mL/cm².

As with previous embodiments of drug delivery systems, permeation enhancing components can be included. Additionally, a heating device can be configured for application over the transdermal patch, including exothermic chemical heating devices, electric heating devices, radiation-based heating devices, etc., all as described above.

As mentioned, although the drug delivered through transdermal absorption will eventually end up in the systemic circulation, a portion of the drug permeated across the skin adjacent to the target tissues is expected to enter the target tissues without passing through the systemic circulation first. This mechanism allows a sufficient amount of the drug to enter the target tissues while producing systemic drug concentrations that are much lower than that produced by typical effective oral products containing the same drug or even other transdermal systems that rely primarily on systemic delivery.

In one embodiment, the target tissues are tissues in or around the knee. Drug molecules delivered across the skin adjacent to the knee, especially the area just above and just below the patella, have good chances to enter the target tissues directly. Drug molecules delivered across the skin sites too far from the knee have lower chances to reach the target tissues but will contribute to the systemic drug concentration (which one wants to minimize) just as much, or more.

The heating device and the drug component, i.e. transdermal patch, can be in one integrated system or in separate units but combined prior to or during use. However, an integrated system can need special designs for addressing issues unique to integrated systems. One of a potential need in an integrated system is prevention of drug migration into the heating device.

In accordance with the present disclosure, osteoarthritis can be treated using the drug delivery systems discussed herein. In a specific embodiment, a method for treating osteoarthritis can include placing a transdermal patch at a human skin site and heating the transdermal patch and human skin with a heating device. The transdermal patch can include a sufficient amount of anti-inflammatory drug for sustained transdermal delivery. The heating device can be configured for heating the human skin site to a specific temperature range from 36° C. to 42° C. The heating device can further be configured for maintaining the temperature of the human skin site within the specific temperature range for a period of at least 5 hours. In one aspect, the transdermal patch can be in contact with the human skin site for a substantially continuous period of time of about 5 hours to about 10 hours.

The systems of the present disclosure can be formulated and configured so to provide therapeutically effective delivery rates of ketoprofen, diclofenac, COX-2 inhibitors, etc., to a subject via transdermally delivery. In one embodiment, the transdermal delivery can specifically be through the skin surrounding the knee. To provide one example, ketoprofen can be considered for various dosages to be delivered at or around the active skin site of the knee joint. In this example, the therapeutically effective delivery of ketoprofen can be achieved through the use of a system which includes a transdermal patch having a skin-drug contact area configured for adhesion to human skin. In one embodiment, the system can be configured to produce a mean blood plasma concentration of ketoprofen in a human subject of at least 33 ng/ml within four hours after initial application of the patch's skin-drug contact area to a skin surface, e.g. the skin surface surrounding a knee. In another embodiment, the mean blood plasma concentration of ketoprofen in the human subject can be at least 40 ng/ml within four hours after initial application of the patch. In another embodiment, the mean blood plasma concentration of ketoprofen in the human subject can be at least 66 ng/ml within six hours after initial application of the patch. In another embodiment, the mean blood plasma concentration of ketoprofen in the human subject can be at least 79 ng/ml within six hours after initial application of the patch. In a further embodiment, the mean blood plasma concentration of ketoprofen in the human subject can be at least 81 ng/ml within eight hours after initial application of the patch. In yet another embodiment, the mean blood plasma concentration of ketoprofen in the human subject can be at least 97 ng/ml within eight hours after initial application of the patch. In another embodiment, the mean blood plasma concentration of ketoprofen in the human subject can be at least 92 ng/ml within ten hours after initial application of the patch. In another embodiment, the mean blood plasma concentration of ketoprofen in the human subject can be at least 110 ng/ml within ten hours after initial application of the patch. The system can deliver ketoprofen in at a rate such that the peak blood plasma concentration of ketoprofen in the patient occurs at from 8-14 hours after initial administration of the patch to the patient's skin surface. In one embodiment, the peak blood plasma concentration of ketoprofen in the subject can occur at about 9-13 hours after initial administration of the patch to the skin surface of the patient.

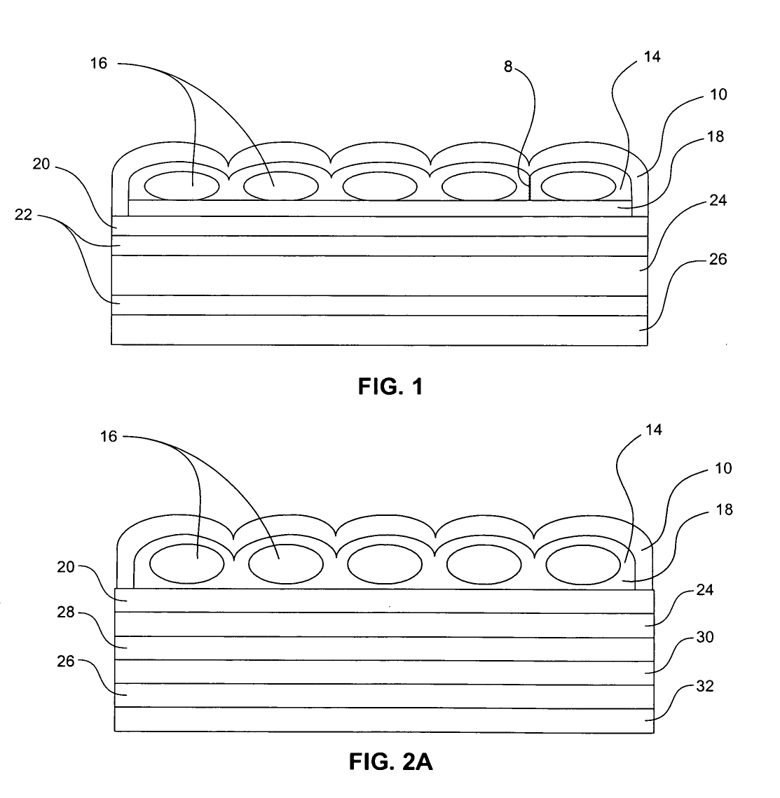

By way of example, regardless of the drug choice, FIG. 1 is a profile of one embodiment that illustrates one configuration of a device that can be used in accordance with embodiments of the present disclosure. The layers incorporated into one embodiment of the present disclosure include a stretchable polymeric air-impermeable foam or elastic material layer 10 with holes (not shown in this view) for allowing air to pass therethrough, heating elements comprising an air permeable enclosure 14 containing exothermic heating composition 16, a polymeric layer 18 that can be used to prevent transfer of water and salt, a layer of transfer adhesive 20, films of poly(ethylene acrylic acid) 22, a thin metal layer 24, such as a foil, and the drug-containing adhesive layer 26. A release liner (not shown in this embodiment) can be present to protect the drug-containing adhesive layer, as is known in the art. Alternative configurations are also useable.

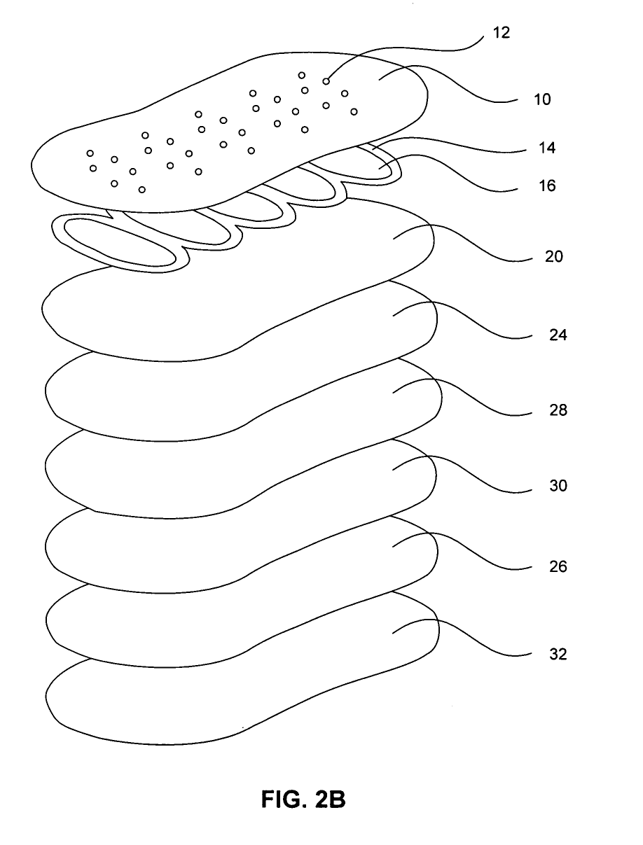

Alternatively, FIG. 2B sets forth an alternative exploded view of an embodiment of the present disclosure that is similar to the embodiment in FIG. 2A, and slightly different than the embodiment shown in FIG. 1. The layers incorporated into this embodiment include a stretchable polymeric air-impermeable foam or elastic material layer 10 with holes 12 for allowing air to pass therethrough. Heating elements are present and can comprise an air permeable enclosure 14 containing exothermic heating composition 16. A thin metal layer 24, such as a foil, is positioned immediately adjacent to a transfer adhesive 20 (such as an acrylic transfer layer), which can join the thin metal layer to the heating elements. One or two of polymeric layers 28, 30, can also optionally be present, such as ethyl acrylic acid and polyethylene, respectively, which are positioned between the thin metal layer and a drug-containing adhesive layer 26. A release liner 32, is also shown in this embodiment.

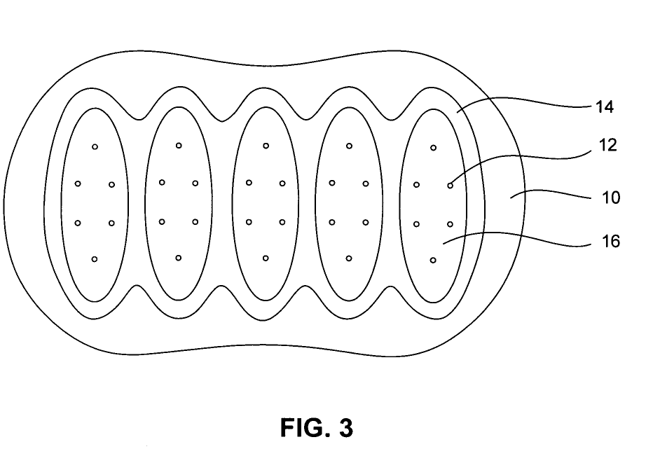

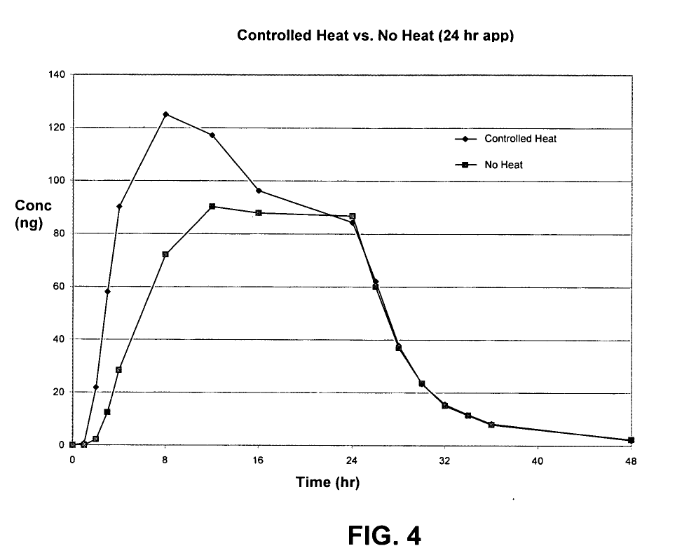

FIG. 3 shows an exemplary top view of the device of FIG. 1 or FIG. 2. In this embodiment, the stretchable polymeric air-impermeable foam or elastic material layer 10 with holes 12 is shown. Additionally, the heating elements, including the air permeable enclosure 14 and the exothermic heating composition 16 are shown as outward facing depression in the elastic material. FIGS. 4-6 are described in greater detail in the following examples.

### EXAMPLES

The following examples illustrate the embodiments of the disclosure that are presently best known. However, it is to be understood that the following is only exemplary or illustrative of the application of the principles of the present disclosure. Numerous modifications and alternative compositions, methods, and systems may be devised by those skilled in the art without departing from the spirit and scope of the present disclosure. The appended claims are intended to cover such modifications and arrangements. Thus, while the present disclosure has been described above with particularity, the following example provides further detail in connection with what is presently deemed to be the most practical and preferred embodiments of the disclosure.

### Example 1

### Heating Devices Adapted for Use with Ketoprofen Transdermal Patches

An exothermic heating device (with appropriate ketoprofen drug matrix patch associated therewith) that is configured similarly to those shown in FIG. 1 or 2 can be prepared in accordance with Table 1, as follows:

TABLE 1

| Component | Device 1 | | Device 2 | |
|---|---|---|---|---|
| Active Pharmaceutical Ingredient: | | | | |
| Drug Patch Area | 100 | cm² | 172 | cm² |
| Ketoprofen, USP (weight %) | 79 | mg (21%) | 136 | mg (21%) |
| Excipients: | | | | |
| Acrylic Adhesive | 297 | mg (79%) | 511 | mg (79%) |
| Films: | | | | |
| Top Cover Film with Holes (Occlusive | 156 | cm² | 172 | cm² |
| film for temperature regulation) | | | | |
| Formable Web (Reservoir for powder | NA | | 104 | cm² |
| dosing) | | | | |
| Heat Sealable Film (Contain heating | NA | | 104 | cm² |
| powder) | | | | |
| Filter Paper (Contain heating powder) | 103 | cm² | NA | |
| Bottom film (Occlusive film and skin | 156 | cm² | NA | |
| contact layer) | | | | |
| Barrier Film Layer | | | | |
| Ethylene Acrylic Acid - 0.5 mil (Non- | NA | | 172 | cm² |
| barrier substrate for lamination of | | | | |
| transfer adhesive) | | | | |
| 35 gauge foil (Barrier film to isolate drug | NA | | 172 | cm² |
| layer) | | | | |
| White Ethylene Acrylic Acid - 0.5 mil | NA | | 172 | cm² |
| (Tie layer to bond polyethylene film to | | | | |
| foil) | | | | |
| Polyethylene Film - 1 mil (Substrate for | NA | | 172 | cm² |
| lamination of drug-in-adhesive matrix) | | | | |
| Polyester Release Liner - 2 mils | NA | | 172 | cm² |
| (Removable liner for patient to remove | | | | |
| when applying patch) | | | | |
| Heating Pockets | | | | |
| Number of Pockets | 6 | | 5 | |
| Pocket Shape | Rectangle | | Oval | |
| Area of Pockets | 15.7 | cm² | 12.4 | cm² |
| Pocket Dimensions | 3.2 cm × 4.9 cm | | 2.5 cm × 6.3 cm | |
| Pocket fill weight | 3.5 | g | 4.5 | g |
| Iron Powder (weight %) | 2.15 | g/pocket (61.5%) | 2.81 | g/pocket (62.5%) |
| Activated Carbon (weight %) | 0.68 | g/pocket (19.3%) | 0.56 | g/pocket (12.5%) |
| Flour Salt (weight %) | 0.27 | g/pocket (7.7%) | 0.79 | g/pocket (17.5%) |
| Vermiculite (weight %) | 0.40 | g/pocket (11.5%) | 0.34 | g/pocket (7.5%) |
| Water Dosing Solution | | | | |
| Sodium Sulfite (weight %) | NA | | 0.125 | g/pocket (9.9%) |
| Liquinox (weight %) | NA | | 0.006 | g/pocket (0.5%) |
| Water, USP (weight %) | 1.2 | g/pocket (100%) | 1.129 | g/pocket (89.6%) |
| Heating Patch in General | | | | |
| Patch Area | 156 | cm² | 172 | cm² |
| Total Pod Area | 103 | cm² | 104 | cm² |

| | | |
|---|---|---|
| Total Pod Dimensions | 15.9 cm × 6.5 cm | 15.9 cm × 7.1 cm |

### Example 2

### Improved Benefits of Heat with Dermal Drug Delivery

Ketoprofen patches prepared as described as Device 1 above were administered to the back area of two groups of human subjects. One group (13 subjects) received the patch without heating. The other group (12 subjects) received the patch with an exothermic heating device that kept the mean skin temperature in the range of 36° C. to 42° C. for more than 6 hours (heating area: approximately 94 cm², which is similar to that described in FIGS. 1-3). The weight of the entire system placed on the subjects in the heated group, including the drug component and heating device, weighed about 34.5 grams. Concentrations of ketoprofen in blood samples taken at specific time intervals were measured and are shown in FIG. 4 (mean of all subjects in each group). Although the target is the tissues of the knee, drug concentrations in blood circulation are believed to be a good measure of how much drug is delivered across the skin. As can be seen in FIG. 4, heating to the specific temperature range of 38° C. to 42° C. for more than 6 hours significantly increased the transdermal delivery of ketoprofen, especially in the early hours. Although the test was conducted on the back skin, an intended application site of the product is the skin area adjacent to the knee or other similar joint. The skin site difference is not expected to cause material difference in plasma ketoprofen concentrations or the effect of heat. The systemic ketoprofen concentrations (ketoprofen concentrations in plasma) are used to gauge the transdermal ketoprofen permeability and the effect of heat for the aforementioned reasons.

### Example 3

### Improved Benefits of Heat with Dermal Drug Delivery

A ketoprofen patch with 172 cm² surface area prepared as shown as Device 2 above is placed on the skin area just above the patella of a human subject suffering from osteoarthritis of the knee. The patch includes an exothermic heating device that can keep the mean skin temperature of the skin in the range of 36° C. to 42° C. for more than 6 hours (approximately 62 cm² heating area). The weight of the entire system placed on the subject is about 36.7 grams. A sufficient amount of the drug will enter the knee tissue of the subject to cause significant reduction of the pain score reported by the subject, which is superior to that which would occur if the ketoprofen patch were applied to the knee without the heating device.

### Example 4

### Heating Profiles

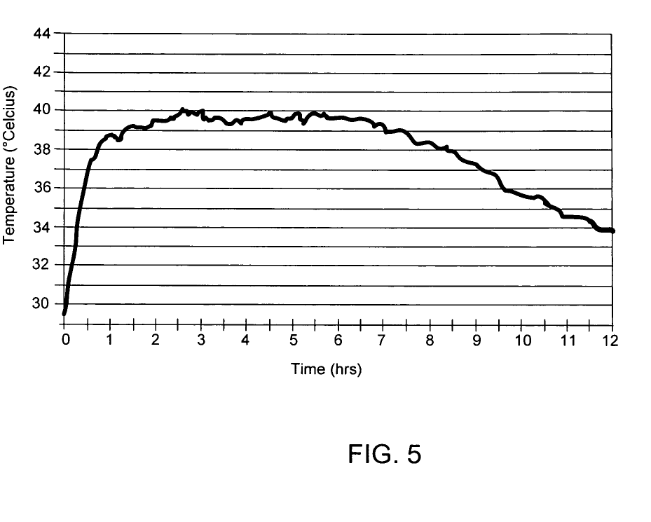

Several devices prepared in accordance with Device 2 above (without drug) was placed on the knees of a small group of subjects, and the skin temperature over 12 hours was obtained and characterized in FIG. 5. As can be seen, the skin temperature profile for this group ranged from 36° C. to 42° C. for about 9 hours.

### Example 5

### Heating Profiles

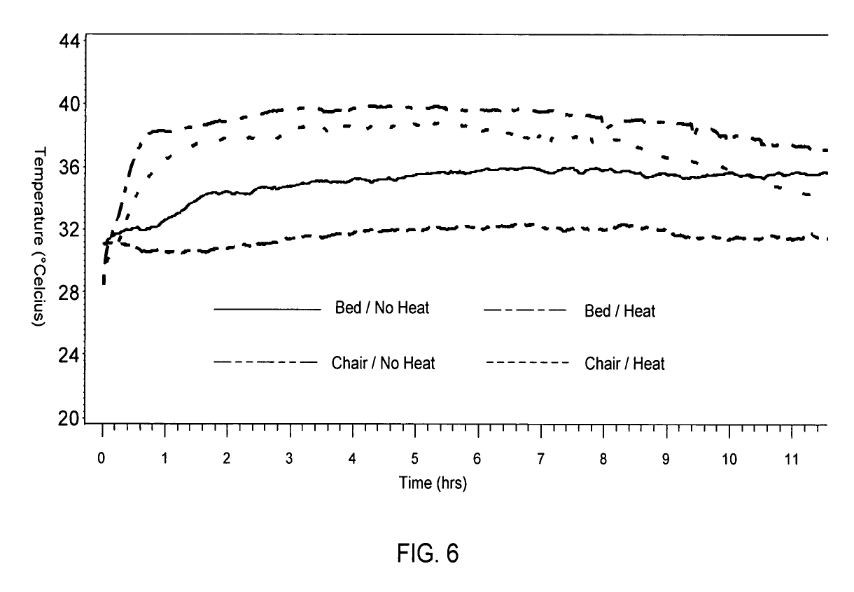

Several devices prepared in accordance with Device 2 above (without drug) were prepared and tests were conducted on 32 knees with the device and 32 knees without the device. Specifically, 4 groups of 16 knees were evaluated under different conditions for comparison purposes. Specifically, to 16 knees was applied the device, which was left uncovered as the subject sat in a chair for at least 12 hours (subject wearing shorts) (Referred to as “Chair/Heat”) to simulate conditions where maximum exposure to the ambient air would occur. To 16 knees was applied the device, which was covered by a light sheet and blanket (Referred to as “Bed/Heat”) to simulate normal overnight usage. As a control, 16 knees were measured for temperature without heat applied to the knee (Referred to as “Chair/No Heat”), and 16 knees were measured for temperature without applied heat while the subjects' knees were covered by a light sheet and blanket (Referred to as “Bed/No Heat). The data collected is provided in FIG. 6. As can be seen, the skin temperature profile for this group ranged from 36° C. to 42° C. for at least 8 hours, whether the device was subject to ambient air or covered by a light sheet and blanket.

### Example 6

### Heating Devices Adapted for Use with Diclofenac Transdermal Patches

Transdermal patch devices are prepared in accordance with Example 1 (Device 1 and Device 2), except that diclofenac is used rather than ketoprofen. It is noted that diclofenac is less skin permeable using the Example 1 formulations, and generally, the drug is more potent than ketoprofen. Thus, adjustment of the drug concentration can be carried out in order to achieve a desired therapeutic effect.

### Example 7

### Ketoprofen Plasma Levels Achieved by on Knee Drug Delivery

Ketoprofen patches (including heating element) containing 136 mg ketoprofen (surface area 172 cm²) were applied above the knee to 24 human subjects during two study sessions. In one study session, subjects received one patch above one knee and in the other study session subjects received two patches simultaneously, one above each knee. The patches were applied for 12 hours and venous blood samples were collected during both study session.

Ketoprofen blood plasma levels of the study participants were measured at two hour time intervals for the first 24 hours and then at 4 hour time intervals for hours 24-36 and at 6 hour time intervals from 36-48. The mean measurement for each the times period are set forth in Table 2.

TABLE 2

| | | | Two Patches |
|---|---|---|---|
| | | | Dose-Normalized |
| | Single Patch | Two Patches | to one patch |
| Time point | Mean Ketoprofen Plasma Concentration (ng/ml) | | |
| (hrs) | (N = 22) | | |
| 2 | 13.5 | 7.8 | 3.9 |
| 4 | 62.9 | 89.2 | 44.6 |
| 6 | 96.7 | 176.2 | 88.1 |
| 8 | 116.1 | 216.6 | 108.3 |
| 10 | 132.5 | 245.6 | 122.8 |
| 12 | 120.9 | 228.7 | 114.3 |
| 14 | 79.8 | 156.3 | 78.1 |
| 16 | 48.5 | 96.3 | 48.2 |
| 18 | 36.3 | 68.4 | 34.2 |
| 20 | 27.6 | 52.2 | 26.1 |
| 24 | 16.4 | 31.0 | 15.5 |
| 28 | 10.0 | 18.6 | 9.3 |
| 32 | 6.1 | 12.3 | 6.2 |
| 36 | 4.9 | 9.9 | 5.0 |
| 42 | 3.9 | 7.2 | 3.6 |
| 48 | 3.2 | 6.2 | 3.1 |

Ketoprofen Plasma concentrations generated by each square centimeter of the patch (plasma ketoprofen concentrations in above table divided by surface areas of patch(es) applied to the knee(s))

TABLE 3

| | |
|---|---|
| Single Patch | Two Patches |

| | |
|---|---|
| | Mean Ketoprofen Plasma |
| | Concentration produced by unit skin- |
| Time point | drug contact area ((ng/ml/cm²) |
| (hrs) | (N = 22) |

| | | |
|---|---|---|
| 2 | 0.078 | 0.023 |
| 4 | 0.366 | 0.259 |
| 6 | 0.562 | 0.512 |
| 8 | 0.675 | 0.630 |
| 10 | 0.770 | 0.714 |
| 12 | 0.703 | 0.665 |
| 14 | 0.464 | 0.454 |
| 16 | 0.282 | 0.280 |
| 18 | 0.213 | 0.199 |
| 20 | 0.160 | 0.152 |
| 24 | 0.095 | 0.090 |
| 28 | 0.058 | 0.054 |
| 32 | 0.035 | 0.036 |
| 36 | 0.028 | 0.029 |
| 42 | 0.023 | 0.021 |
| 48 | 0.019 | 0.018 |

While the disclosure has been described with reference to certain preferred embodiments, those skilled in the art will appreciate that various modifications, changes, omissions, and substitutions can be made without departing from the spirit of the disclosure. It is therefore intended that the invention be limited only by the scope of the appended claims.

What is claimed is:

1. A system for delivering an anti-inflammatory drug, comprising:
a transdermal patch including a sufficient amount of anti-inflammatory drug for sustained transdermal delivery at a human skin site; and
a heating device configured for
i) application over the transdermal patch and the human skin site,
ii) heating the human skin site to a specific temperature range from 36° C. to 42° C., and
iii) maintaining the human skin site within the specific temperature range for a period of at least 5 hours.

2. The system of claim 1, wherein the anti-inflammatory drug is a non-steroidal anti-inflammatory drug.

3. The system of claim 1, wherein the anti-inflammatory drug is ketoprofen.

4. The system of claim 1, wherein the anti-inflammatory drug is diclofenac.

5. The system of claim 1, wherein the anti-inflammatory drug is a COX-2 inhibitor.

6. The system of claim 1, wherein the anti-inflammatory drug is a COX-3 inhibitor.

7. A system as in claim 1, wherein the heating device is configured to heat the human skin site to said specific temperature range within 60 minutes of application and activation, and once within said specific temperature range, is configured to maintain the human skin site within said specific temperature range for the at least 5 hours.

8. A system as in claim 1, wherein the heating device is configured to heat the human skin site to said specific temperature range within 45 minutes of application and activation, and once within said specific temperature range, is configured to maintain the human skin site within said specific temperature range for the at least 5 hours.

9. A system as in claim 1, wherein the heating device is configured to heat the human skin site to said specific temperature range within 30 minutes of application and activation, and once within said specific temperature range, is configured to maintain the human skin site within said specific temperature range for the at least 5 hours.

10. A system as in claim 1, wherein the heating device is configured to heat the human skin site to said specific temperature range within 20 minutes of application and activation, and once within said specific temperature range, is configured to maintain the human skin site within said specific temperature range for the at least 5 hours.

11. A system as in claim 1, wherein the heating device is configured to heat the human skin site to said specific temperature range no faster than 8 minutes from application and activation.

12. A system as in claim 1, wherein the heating device is configured to heat the human skin site to said specific temperature range no faster than 10 minutes of application and activation.

13. A system as in claim 1, wherein the heating device is configured to heat the human skin site to said specific temperature range no faster than 12 minutes of application and activation.

14. A system as in claim 1, wherein the heating device is configured to maintain the human skin site within said specific temperature range for the at least 6 hours.

15. A system as in claim 1, wherein the heating device is configured to maintain the human skin site within said specific temperature range for the at least 7 hours.

16. A system as in claim 1, wherein the heating device is configured to heat the human skin site at an average rate of about 0.1° C./minute to about 1.0° C./minute within 60 minutes of application and activation until the temperature of the skin exceeds about 37° C.

17. A system as in claim 1, wherein the heating device is configured to heat the human skin site at an average rate of about 0.2° C./minute to about 0.80° C./minute within 60 minutes of application and activation until the temperature of the skin exceeds about 37° C.

18. A system as in claim 1, wherein the heating device is configured to raise the temperature of the human skin site at a substantially constant rate until the temperature of the skin exceeds 37° C.

19. A system as in claim 1, wherein the heating device is configured to heat the human skin site at a rate not varying by more than about 0.8° C./minute to the specific temperature range.

20. A system as in claim 1, wherein the heating device is configured to heat the human skin site at a rate not varying by more than about 0.3° C./minute to the specific temperature range.

21. A system as in claim 1, wherein the heating device automatically cools to below 36° C. after heating the human skin site for a period of at least 7 hours within said specific temperature range.

22. A system as in claim 1, wherein the heating device automatically cools to below 36° C. after heating the human skin site for between 5 and 10 hours within said specific temperature range.

23. A system as in claim 1, wherein the human skin site is heated to a temperature of between 38° C. and 42° C. for the period of at least 5 hours.

24. A system as in claim 1, wherein the human skin site is heated to a temperature of between 36° C. and 40° C. for the period of at least 5 hours.

25. A system as in claim 1, wherein the heating device is configured to produce heat from a chemical-based reaction.

26. A system as in claim 25, wherein the chemical-based reaction is an exothermic chemical reaction.

27. A system as in claim 26, wherein the exothermic chemical reaction is an oxidation reaction.

28. A system as in claim 27, wherein oxidation reaction includes an iron oxidation reaction.

29. A system as in claim 25, wherein the chemical based reaction is an oxidation reaction of an alcohol having one to four carbons.

30. A system as in claim 26, wherein oxygen flow for the exothermic chemical reaction is regulated by holes in an air-impermeable cover.

31. A system as in claim 1, wherein the heating device is an electric heating device.

32. A system as in claim 31, wherein the electric heating device is battery operated.

33. A system as in claim 31, wherein the electric heating device is powered by alternating electric current.

34. A system as in claim 1, wherein the heating device includes no more than 35 grams of an exothermic chemical composition, and is configured to heat an area of skin greater than about 40 cm².

35. A system as in claim 34, wherein the exothermic chemical composition is configured to produce all heat for the heating device.

36. A system as in claim 1, wherein the heating device is configured to heat an area of skin greater than about 60 cm².

37. A system as in claim 1, wherein the heating device is configured to heat an area of skin greater than about 80 cm².

38. A system as in claim 1, wherein the heating device is configured to heat an area of skin greater than about 100 cm².

39. A system as in claim 1, wherein the heating device is configured to heat an area of skin greater than about 120 cm².

40. A system as in claim 1, wherein the heating device is configured to heat an area of skin greater than about 40 cm² and is configured to produce a heat variation in the area of skin surface covered by the heating device of less than about 4° C. while maintaining the human skin site within the specific temperature range for a period of at least 5 hours.

41. A system as in claim 1, wherein the heating device is configured to heat an area of skin greater than about 60 cm² and is configured to produce a heat variation in the skin covered by the heating device of less than about 4° C. while maintaining the human skin site within the specific temperature range for a period of at least 5 hours.

42. A system as in claim 1, wherein the heating device is configured to heat an area of skin greater than about 40 cm² and is configured to produce a heat variation in the skin covered by the heating device of less than about 4° C. while maintaining the human skin site within the specific temperature range for a period of at least 7 hours.

43. A system as in claim 1, wherein the heating device is configured to heat an area of skin greater than about 60 cm² and is configured to produce a heat variation in the skin covered by the heating device of less than about 4° C. while maintaining the human skin site within the specific temperature range for a period of at least 5 hours, wherein said heating device also comprises less than about 40 grams of exothermic chemical composition.

44. A system as in claim 1, wherein the heating device comprises a plurality of discrete heating elements.

45. A system as in claim 44, wherein the heating device includes from 2 to 10 heating elements.

46. A system as in claim 44, wherein the heating device includes from 3 to 7 heating elements.

47. A system as in claim 44, wherein the heating elements are arranged in a single line.

48. A system as in claim 44, wherein the heating elements are pouches containing a chemical mixture capable of reacting exothermically.

49. A system as in claim 1, wherein the transdermal patch and the heating device are combined as an integrated unit.

50. A system as in claim 1, configured for application to a joint.

51. A system as in claim 1, configured for application to a knee.

52. A system as in claim 1, wherein the transdermal patch further includes a second anti-inflammatory drug.

53. A system as in claim 1, wherein the transdermal patch includes ketoprofen and a second anti-inflammatory drug.

54. A system as in claim 1, wherein the transdermal patch has a skin contact area where the anti-inflammatory drug is delivered to the skin of at least 60 cm² and the heating device has a weight of less than 100 g.

55. A system as in claim 54, wherein the heating device has a weight of less than 60 g.

56. A system as in claim 54, wherein the heating device has a weight of less than 45 g.

57. A system as in claim 1, wherein the transdermal patch has a skin contact area where the anti-inflammatory drug is delivered to the skin of at least 100 cm² and the entire system has a weight of less than about 50 grams

58. A system as in claim 1, wherein the transdermal patch has a skin contact area where the anti-inflammatory drug is delivered to the skin of at least 150 cm² and the entire system has a weight of less than about 50 grams.

59. A system as in claim 57, wherein the heating device has a heating surface area of at least 50 cm².

60. A system as in claim 58, wherein the heating device has a heating surface area of at least 50 cm²

61. A system for delivering ketoprofen, comprising a transdermal patch having a skin-drug contact area of between 50 and 400 cm², said system configured to produce a mean blood plasma concentration of ketoprofen in a group of at least 10 human subjects of at least 33 ng/ml within four hours after initial application of the patch to a skin surface of a human subject.

62. A system as in claim 61, wherein the skin-drug contract area of the patch is between about 50 to about 250 cm².

63. A system as in claim 61, wherein the skin-drug contract area of the patch is less than about 200 cm².

64. A system as in claim 61, wherein the mean blood plasma concentration of ketoprofen in a group of at least 10 human subjects is at least 40 ng/ml within four hours after initial application.

65. A system as in claim 61, wherein the mean blood plasma concentration of ketoprofen in a group of at least 10 human subjects is at least about 66 ng/ml within 6 hours after initial application of the patch to a skin surface of a human subject.

66. A system as in claim 61, wherein the mean blood plasma concentration of ketoprofen in a group of at least 10 human subjects is at least about 79 ng/ml within 6 hours after initial application of the patch to a skin surface of a human subject.

67. A system as in claim 61, wherein the mean blood plasma concentration of ketoprofen in a group of at least 10 human subjects is at least about 81 ng/ml within 8 hours after initial application of the patch to a skin surface of a human subject.

68. A system as in claim 61, wherein the mean blood plasma concentration of ketoprofen in a group of at least 10 human subjects is at least about 97 ng/ml within 8 hours after initial application of the patch to a skin surface of a human subject.

69. A system as in claim 61, wherein the mean blood plasma concentration of ketoprofen in a group of at least 10 human subjects is at least about 92 ng/ml within 10 hours after initial application of the patch to a skin surface of a human subject.

70. A system as in claim 61, wherein the mean blood plasma concentration of ketoprofen in a group of at least 10 human subjects is at least about 110 ng/ml within 10 hours after initial application of the patch to a skin surface of a human subject.

71. A system as in claim 61, wherein the peak mean plasma concentration of ketoprofen occurs at from about 8 hours to about 12 hours after initial administration of the patch to a skin surface of a human subject.

72. A system as in claim 61, wherein the system further comprises a heating device.

73. A system as in claim 72, wherein the heating device is configured for
i) application over the transdermal patch and the human skin site,
ii) heating the human skin site to a specific temperature range from about 36° C. to about 42° C., and
iii) maintaining the human skin site within the specific temperature range for a period of at least 5 hours.

74. A system as in claim 61, wherein the system further comprises a method of enhancing ketoprofen skin permeation, selected from the group of chemical enhancement, iontophoresis, infrared radiation, and ultrasound.

75. A system for transdermally delivering ketoprofen, comprising a transdermal patch having a skin-drug contact area of between 50 and 400 cm², said system configured to produce a plasma ketoprofen concentration produced by unit skin-drug contact area in a group of at least 10 human subjects of at least 0.19 ng/ml/cm² within four hours after initial application of the patch to a skin surface of a human subject.

76. A system as in claim 75, wherein the mean blood plasma concentration of ketoprofen produced by unit skin-drug contact area in a group of at least 10 human subjects is at least 0.23 ng/ml/cm² within four hours after initial application.

77. A system as in claim 75, wherein the mean blood plasma concentration of ketoprofen produced by unit skin-drug contact area in a group of at least 10 human subjects is at least 0.38 ng/ml/cm² within six hours after initial application.

78. A system as in claim 75, wherein the mean blood plasma concentration of ketoprofen produced by unit skin-drug contact area in a group of at least 10 human subjects is at least 0.47 ng/ml/cm² within six hours after initial application.

79. A system as in claim 75, wherein the mean blood plasma concentration of ketoprofen produced by unit skin-drug contact area in a group of at least 10 human subjects is at least 0.47 ng/ml/cm² within eight hours after initial application.

80. A system as in claim 75, wherein the mean blood plasma concentration of ketoprofen produced by unit skin-drug contact area in a group of at least 10 human subjects is at least 0.56 ng/ml/cm² within eight hours after initial application.

81. A system as in claim 75, wherein the mean blood plasma concentration of ketoprofen produced by unit skin-drug contact area in a group of at least 10 human subjects is at least 0.53 ng/ml/cm² within ten hours after initial application.

82. A system as in claim 75, wherein the mean blood plasma concentration of ketoprofen produced by unit skin-drug contact area in a group of at least 10 human subjects is at least 0.64 ng/ml/cm² within ten hours after initial application.

83. A system as in claim 75, wherein the system further comprises a method of enhancing ketoprofen skin permeation, selected from the group of chemical enhancement, iontophoresis, infrared radiation, and ultrasound.

84. A system as in claim 75, wherein the peak plasma concentration of ketoprofen occurs at from about 7 hours to about 14 hours after administration of the patch to a skin surface of a human subject.

85. A system as in claim 75, wherein the transdermal patch is formulated to be left on the skin surface of a human subject for a period of 8 hours to about 14 hours.

86. A system as in claim 75, wherein the transdermal patch is formulated to be left on the skin surface of a human subject for a period of about 10 to about 12 hours.

87. A system as in claim 75, wherein the transdermal patch is formulated to be left on the skin surface of a human subject for a period of about 12 hours.

88. A system as in claim 75, wherein the system further comprises a heating device.

89. A system as in claim 75, wherein the heating device is configured for
i) application over the transdermal patch and the human skin site,
ii) heating the human skin site to a specific temperature range from about 36° C. to about 42° C., and
iii) maintaining the human skin site within the specific temperature range for a period of at least 5 hours.

90. A system as in claim 75, wherein the heating device is capable of generating heat using oxidation of metal powder.

91. A system as in claim 75, wherein the heating device is integrated with the transdermal patch.

92. A system as in claim 75, wherein the system is configured for application to a human knee and mean blood plasma concentration of ketoprofen is achieved by delivery through the skin of the human knee.

93. A system for delivering ketoprofen, comprising a transdermal patch having a skin-drug contact area and a heating device, said system capable of producing a mean blood plasma concentration of ketoprofen in human subjects that does not peak within 7 hours of commencement of the application to the skin surface of human subjects.

94. A system as in claim 93, said system capable of producing a mean blood plasma concentration of ketoprofen in human subjects that does not peak within 8 hours of commencement of application to the skin surface of human subjects.

95. A system for delivering ketoprofen, comprising a transdermal patch having a skin-drug contact area and a heating device, said system capable of producing a mean blood plasma concentration of ketoprofen in human subjects that exceeds about 74 ng/mL within 7 hours of commencement of application to the skin surface of human subjects.

96. A system for delivering ketoprofen, comprising a transdermal patch having a skin-drug contact area and a heating device, said system capable of producing a mean blood plasma concentration of ketoprofen in human subjects that exceeds about 81 ng/mL within 8 hours of commencement of application to the skin surface of human subjects.

97. A system for delivering diclofenac, comprising a transdermal patch having a skin-drug contact area and including a sufficient concentration of diclofenac for sustained transdermal delivery at the skin-drug contact area, said transdermal patch capable of producing a blood plasma concentration of diclofenac per unit of the skin-drug contact area within 4 hours of application to a skin surface of a human subject of at least 0.08 ng diclofenac/mL/cm².

98. A system as in claim 97, wherein the system is capable of producing a mean blood plasma concentration of diclofenac per unit of the skin-drug contact area in human subjects within 4 hours of application to the skin surface of human subjects of at least about 0.11 ng diclofenac/mL/cm².

99. A system as in claim 97, wherein the system is capable of producing a mean blood plasma concentration of diclofenac per unit of the skin-drug contact area in human subjects within 8 hours of application to the skin surface of human subjects of at least about 0.23 ng diclofenac/mL/cm².

100. A system as in claim 97, wherein the system further comprises a permeation enhancing component other than heat.

101. A system as in claim 97, wherein the system further comprises a heating device.

102. A system as in claim 101, wherein the heating device is configured for
i) application over the transdermal patch and the human skin site,
ii) heating the human skin site to a specific temperature range from about 36° C. to about 42° C., and
iii) maintaining the human skin site within the specific temperature range for a period of at least 5 hours.

103. A system as in claim 101, wherein said heating device is capable of generating heat using oxidation of metal powder.

104. A system as in claim 101, wherein the heating device is adapted within a heating device configured for application over the transdermal patch.

105. A system as in claim 101, wherein the heating device is integrated with the transdermal patch.

106. A method for treating osteoarthritis, comprising:
placing a transdermal patch at a human skin site adjacent to a joint suffering from osteoarthritis, said transdermal patch comprising a sufficient amount of an anti-inflammatory drug for sustained transdermal delivery for at least 6 hours; and
heating the transdermal patch and human skin with a heating device configured for:
i) heating the human skin site to a specific temperature range from 36° C. to 42° C., and
ii) maintaining the human skin site within the specific temperature range for a period of at least 5 hours.

107. A method as in claim 106, wherein the anti-inflammatory drug is a non-steroidal anti-inflammatory drug.

108. A method as in claim 106, wherein the anti-inflammatory drug is diclofenac.

109. A method as in claim 106, wherein the anti-inflammatory drug is a COX-2 inhibitor.

110. A method as in claim 106, wherein the anti-inflammatory drug is a COX-3 inhibitor.

111. A method as in claim 106, wherein the anti-inflammatory drug is ketoprofen.

112. A method as in claim 106, wherein the human skin is heated to a temperature of from 38° C. to 42° C.

113. A method as in claim 106, wherein the human skin is heated to a temperature of from 36° C. to 40° C.

114. A method as in claim 106, wherein the joint is generating pain associated with osteoarthritis.

115. A method as in claim 106, wherein the joint is a knee.

116. A method as in claim 106, wherein the transdermal patch is in contact with the human skin site for a substantially continuous period of about 5 hours to about 14 hours.

117. A method as in claim 106, wherein the heating device and the transdermal patch are integrated.

118. A method as in claim 106, wherein the method further includes the use of a permeation enhancement means for enhancing the permeation of the anti-inflammatory drug into the skin, said means selected from the group consisting of chemical enhancement, iontophoresis, infrared radiation, and ultrasound.

119. A method as in claim 106, wherein the step of maintaining contact is for a period of at least 8 hours, and wherein
a) the mean blood plasma concentration of ketoprofen is at least about 33 ng/ml within four hours after initial application;
b) the mean blood plasma concentration of ketoprofen is at least about 66 ng/ml within 6 hours after initial application; and
c) the mean blood plasma concentration of ketoprofen is at least about 81 ng/ml within 8 hours after initial application.

120. A system for delivering an anti-inflammatory drug, comprising:
a transdermal patch including a sufficient amount of anti-inflammatory drug for sustained transdermal delivery at a human skin site; and
a heating device configured for
i) application over the transdermal patch and the human skin site,
ii) heating the human skin site to a specific temperature range from 36° C. to 42° C., and
iii) maintaining the human skin site within the specific temperature range for a period of at least 5 hours.
wherein the transdermal patch has a formulation-skin contact area of at least 150 cm², and said system weighs no more than 45 grams.
